(19) United States
(12) Patent Application Publication
(10) Pub. No.: US 2022/0008279 A1
(43) Pub. Date: Jan. 13, 2022

(54) BODY WELLNESS DEVICE AND METHOD OF USE

(71) Applicant: Scott Ryan Conrad, San Diego, CA (US)

(72) Inventor: Scott Ryan Conrad, San Diego, CA (US)

(21) Appl. No.: 17/368,600

(22) Filed: Jul. 6, 2021

Related U.S. Application Data
(60) Provisional application No. 63/050,756, filed on Jul. 11, 2020.

(51) Int. Cl. A61G 15/00; A61M 21/02; A61G 13/00; A63B 21/00

(52) U.S. Cl. CPC A61G 15/002; A61M 21/02; A61M 2021/0022; A61G 15/007; A63B 21/4037; A61G 13/009

(57) ABSTRACT

A body wellness device comprising a plurality of rigid panels; one or more hinges coupling the rigid panels together whereby the plurality of rigid panels are foldable in an accordion style fashion into at least a flat coplanar configuration, a chair configuration, and carry configuration.

### CROSS REFERENCE TO RELATED APPLICATION

This application claims the benefit of U.S. Provisional Patent Application No. 63/050,756, filed Jul. 11, 2020, which is incorporated by reference herein.

### FIELD OF THE INVENTION

The present invention relates, in general, to a body wellness device, and, in particular, to a combination exercise board, meditation chair, and sound frequency healing table.

### BACKGROUND OF THE INVENTION

In today's world it is becoming more apparent that different forms of stretching, meditation, sound frequency healing, and breathwork are crucial for the wellbeing of the a human body and mind. As of yet, there is not a stand-alone product that a person can go to for all these aforementioned mind, body, and vibrational needs and practices. To stretch, do yoga, or any sort of physical therapy a user might have a pad, yoga mat, or some other kind of surface they use. For meditation, people generally don't use any device or if they do it's some kind of chair or blanket. Finally, for breathwork and sound healing, which is harmonic resonance with body and sound vibrations, since the popularity is rapidly rising there are many forms of practice being done, but no portable products to aid the user in guided or personal practices.

### SUMMARY OF THE INVENTION

An aspect of the invention solves these problems by providing a body wellness device/board including an all-in-one, portable trifold rigid surface, to do any movement on with the option to add an interchangeable blue-tooth speaker or any frequency generating mechanism that would allow the user to experience sound vibrations close to their body.

Another aspect of the invention involves a body wellness device/board including three rectangular pieces of rigid material that trifold accordion style, and are connected by hinging mechanisms. The unit could also use any pivoting agent or any another connection devices that would allow any of the three pieces to be connected to each other or not. When the unit is folded out completely flat the user can practice any form of movement they desire on it, or when it is partially folded out it can be a chair for meditation or just sitting. Lastly, there is an option for the user to add an interchangeable blue-tooth speaker that allows them to experience sound vibrations close to their body.

In one or more implementations of the aspect of the invention described immediately above, the body wellness board includes a yoga style mat that is attached to one side of the unit that allows the user more comfort in any movement or sitting when on that side. When the unit is flipped over you can use the side that has no yoga mat surface. Also, in an embodiment, for the blue-tooth speaker(s) or other frequency generating devices there are routed out areas in the rigid material where a the sound producing mechanism is placed that generates sound vibrations for the user to enjoy any way they see fit. There are multiple interchangeable slots where the frequency generating device can reside to give the user control of where on their body they want the sound to be closer to. The choice of sound vibrations would be controlled within the unit by an attached mechanism or by digitally being connected to a smart phone, tablet device, computer, or anything else that has frequency sharing, blue-tooth, or wi-fi capabilities. In another embodiment, the speaker or frequency generating device would have a housing or some kind of encasing it to protect from any body interference or outside elements and allow easier transferability among the routed out speaker placement slots on the unit.

Another aspect of the invention involves a body wellness device comprising a plurality of rigid panels; one or more hinges coupling the rigid panels together whereby the plurality of rigid panels are foldable in an accordion style fashion into at least a flat coplanar configuration, a chair configuration, and carry configuration.

In one or more implementations of the aspect of the invention described immediately above, the body wellness device includes one or more of the following: the plurality of rigid panels include three tri-fold panels; the plurality of rigid panels are wood panels; the plurality of rigid panels include a rigid surface configured for a user to one or more of practice movements upon, sit upon, and recline upon; the plurality of rigid panels include a mat surface configured for a user to one or more of practice movements upon, sit upon, and recline upon; a handle configured to be used to carry the body wellness device' the plurality of rigid panels include a lateral side and a slot whereby when the plurality of rigid panels are in the carry configuration, the plurality of slots overlap each other to form a single handle along the lateral side; the plurality of rigid panels include one or more slots configured to receive respective wireless frequency emission devices; the plurality of rigid panels include one or more wireless frequency emission devices received within the one or more slots, a vibroacoustic housing surrounding the one or more slots, and the one or more wireless frequency emission devices are configured to receive signals from a signal generating device and transmit one or more vibration frequencies to the user via the vibroacoustic housing surrounding the one or more slots; one or more rigid inserts received within the one or more slots to cover the one or more slots; the plurality of rigid panels include one or more wireless frequency emission devices, a vibroacoustic housing surrounding the wireless frequency emission devices, and the one or more wireless frequency emission devices are configured to receive signals from a signal generating device and transmit one or more vibration frequencies to the user via the vibroacoustic housing surrounding the one or more wireless frequency emission devices; and/or one or more electromagnetic frequency protectors covering the one or more wireless frequency emission devices.

A further aspect of the invention involves a method of using a body wellness device, the body wellness device comprising a plurality of rigid panels; one or more hinges coupling the rigid panels together whereby the plurality of rigid panels are foldable in an accordion style fashion into at least a flat coplanar configuration, a chair configuration, and carry configuration, the method comprising deploying the plurality of rigid panels in an accordion style fashion by pivoting the plurality of rigid panels relative to each other into at least the flat coplanar configuration, the chair configuration, and the carry configuration.

In one or more implementations of the method aspect of the invention described immediately above, deploying the plurality of rigid panels includes deploying the plurality of rigid panels into the chair configuration where one of the plurality of rigid panels lies flat on a support surface, an adjacent one of the plurality of rigid panels, which forms a back support, is inclined at an angle relative to the flat rigid panel, and a further of the plurality of rigid panels forms an “A” configuration with the adjacent rigid panel with respect to the support surface; the plurality of rigid panels include a lateral side and a slot whereby when the plurality of rigid panels are in the carry configuration, the plurality of slots overlap each other to form a single handle along the lateral side, and deploying the plurality of rigid panels includes deploying the plurality of rigid panels into the carry configuration where the plurality of rigid panels stack together so that the plurality of slots overlap each other to form a single handle along the lateral side; the plurality of rigid panels include one or more slots and a vibroacoustic housing surrounding the one or more slots, and the method further comprising receiving one or more wireless frequency emission devices within the one or more slots, receiving signals by the one or more wireless frequency emission devices from a signal generating device, and transmitting one or more vibration frequencies to the user via the vibroacoustic housing surrounding the one or more slots; and/or the plurality of rigid panels include one or more one or more wireless frequency emission devices and a vibroacoustic housing surrounding the one or more wireless frequency emission devices, and the method further comprising receiving signals by the one or more wireless frequency emission devices from a signal generating device, and transmitting one or more vibration frequencies to the user via the vibroacoustic housing surrounding the one or more slots.

### BRIEF DESCRIPTION OF DRAWINGS

The accompanying drawings, which are incorporated in and form a part of this specification illustrate embodiments of the invention and together with the description, serve to explain the principles of the invention.

FIG. 1 is a perspective view of an embodiment of a body wellness board shown completely folded out unit and with no sound capabilities;

FIG. 2 is a perspective view of another embodiment of a body wellness board shown in a completely folded out or deployed configuration, with an attached yoga mat surface, and with no sound capabilities;

FIG. 3 is a perspective view of another embodiment of a body wellness board shown in a completely folded up, undeployed, transport, or carry configuration;

FIG. 4 is a perspective view of another embodiment of a body wellness board shown in a completely folded out or deployed configuration, with a handle, an attached yoga mat surface, and with sound capabilities;

FIG. 5 is a perspective view of another embodiment of a body wellness board shown in a partially folded, chair configuration and with an attached yoga mat and sound capabilities;

FIG. 6 is an enlarged perspective view of the body wellness board with sound capabilities and shows a routed-out area that houses a blue-tooth speaker and a removable cover for the routed-out area;

FIG. 7 is an enlarged perspective view of the body wellness board similar to FIG. 6 and shows a blue-tooth speaker for the routed-out area;

FIG. 8 is an enlarged front elevational view of the body wellness board of FIGS. 6 and 7, and shows to FIG. 6 and shows a blue-tooth speaker received in one of routed-out rigid material housing slots.

FIG. 9 is a block diagram of an example processing system, by which one or more of the processes described herein, may be executed, according to an embodiment.

### DESCRIPTION OF EMBODIMENT OF THE INVENTION

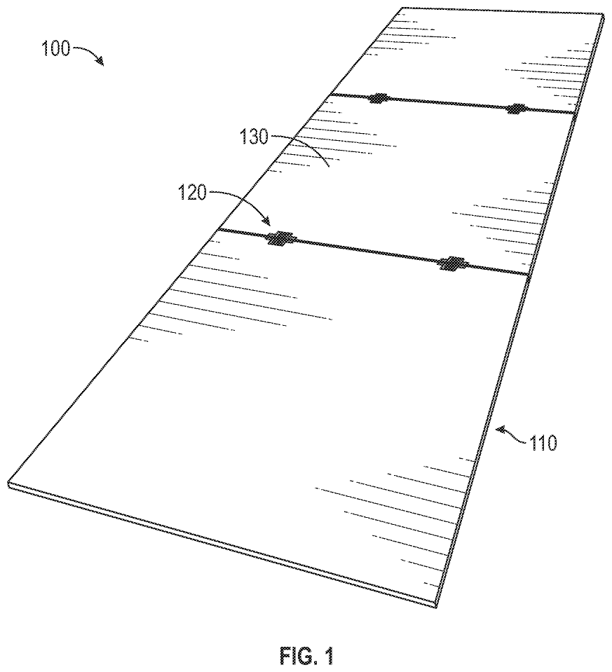

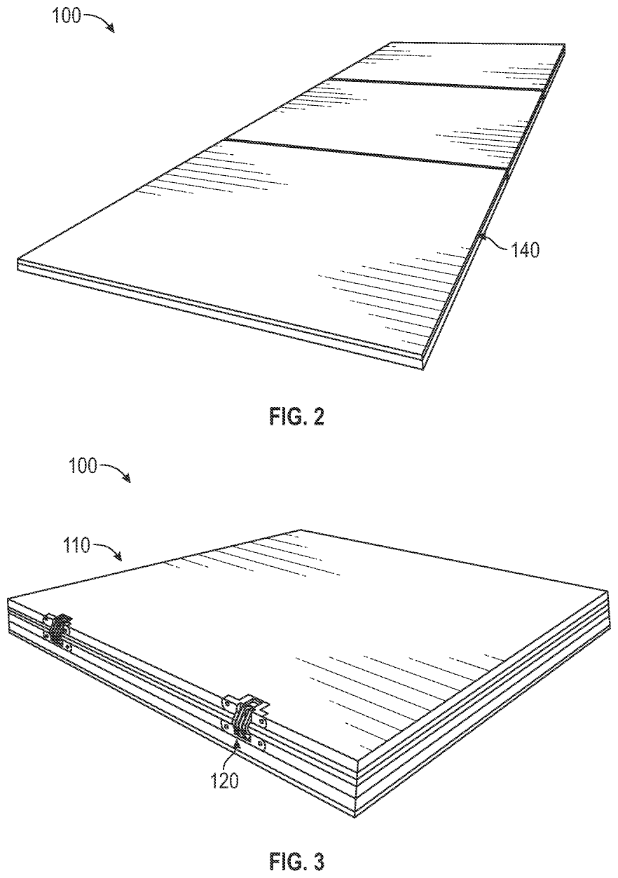

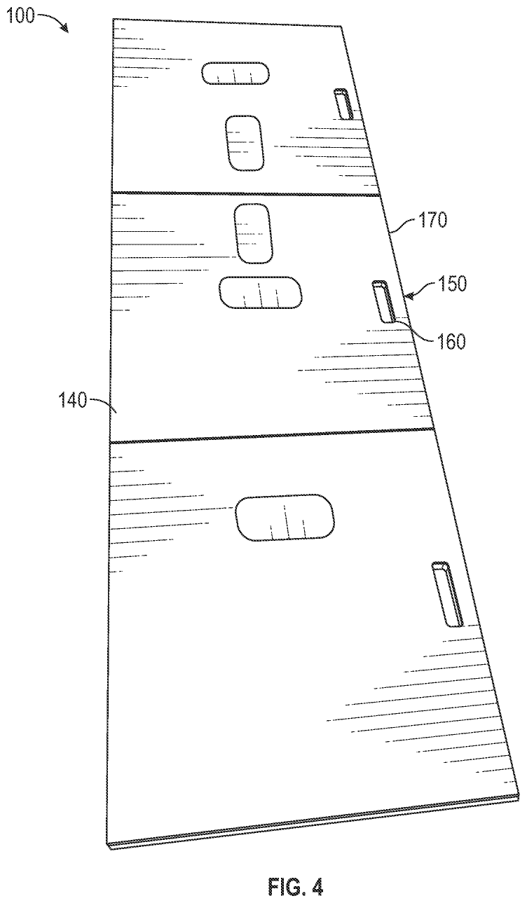

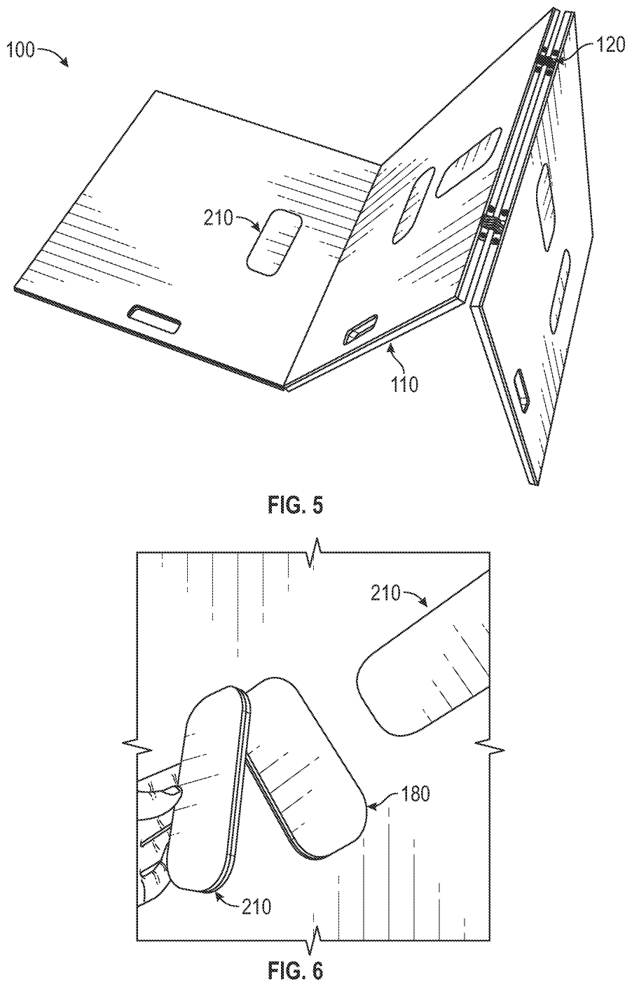

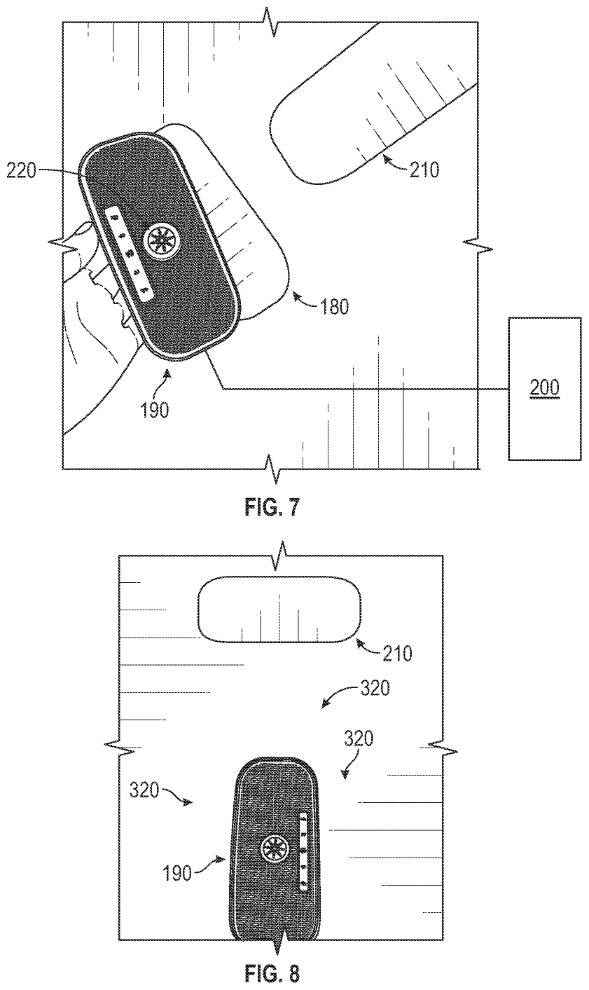

With reference to FIGS. 1-8, a number of embodiments of a body wellness board/device 100 will be described. The body wellness board 100 includes a plurality (e.g., three substantially equally sized) rectangular rigid pieces/panels of wood, plastic, or other rigid material (“panels”) 110 hingeably/pivotally connected by respective hinges, pivoting device/agent, or other connection device 120 that would allow the plurality of rigid panels 110 of the body wellness board 100 to be folded in an accordion style fashion into at least a flat coplanar configuration (FIG. 1), a trifold chair configuration (FIG. 5), and carry configuration (FIG. 3) for easier storage, portability.

The body wellness board 100 includes a rigid surface 130 that a user lies upon, practices movements upon, sits upon, and/or reclines upon. The user may use the body wellness board 100 as shown in FIG. 1, with no additional padding if the user prefers a more rigid surface, or the user could choose to use a yoga mat surface, blanket, or towel on either side for added comfort while doing movement or sitting.

In an alternative embodiment, the body wellness board 100 includes a yoga mat surface 140 attached to the rigid surface 130. In this embodiment, the user has the option of using the body wellness board 100 on the side with the added mat surface 140 or flipping the body wellness board 100 over to use the rigid side with no additional padding.

In another embodiment, each rectangular piece of wood 110 includes a handle 150 formed by a slot 160 adjacent to a lateral side 170. The routed-out handle 150 in this embodiment allows the user to carry the body wellness board 100 with more ease. In this embodiment, when the body wellness board 100 is in a completely folded up, undeployed, transport, or carry configuration, the slots 160 overlap each other to form a single handle 150 for carrying the body wellness board 100.

In a further embodiment, the yoga mat 140 includes a plurality of routed speaker slots 180 that may receive respective wireless frequency emission devices (e.g., blue-tooth speakers) 190, which are controlled by a signal generating device 200 such as mobile device (e.g., iPhone, Android phone, iPad) with software/app for wirelessly controlling frequency emission device(s) 190 or an on-board signal generating device that is part of the body wellness board 100 and with a wired or wireless connection with the frequency emission device(s) 190.

When the wireless frequency emission device(s) 190 are not received in the routed speaker slots 180, a rigid insert material 210 may be received in the routed speaker slots 180 to cover the routed speaker slots 180. The rigid insert material 210 may be made of wood and include the yoga mat/surface 140, which is flush with the surface of the surrounding yoga mat 140, when the rigid insert material 210 is received in the routed speaker slots 180. The wireless frequency emission device(s) 190 may include an electromagnetic frequency sticker or protector 220 thereon to block potential harmful EMF's that a user might otherwise come into contact with. In this embodiment, the user may interchangeably put the frequency emission device(s) 190 in different spots in the body wellness board 100 to feel the frequencies close to the user's body. The interchangeable puzzle piece inserts 210 that are taken out of the board 100 so the frequency emission device 190 can go in are uniformly shaped, so the user can put any piece (e.g., frequency emission device 190, insert 210) in any open spot/slot 180 on the board without having to worry about if they put it in the right slot 180 or not.

Further embodiments of the body wellness board/device 100 includes a portable unit that is the same as or similar to the one described and shown herein and that houses a frequency generating device close to the body so the user can feel the sound vibrations and includes multiple more harmonic resonance/frequency uses.

The body wellness board/device 100 will now be described in use. The body wellness board/device 100 is deployed to the flat configuration in FIG. 1 or the chair configuration in FIG. 5 by pivoting the panels 110 relative to each other via the hinges 120. When the body wellness board/device 100 is folded out completely flat in a coplanar configuration as shown in FIG. 1, the user can practice any form of movement they desire on it, or when it is partially folded out it (where one panel 110 is flat on the ground and the other two panels 110 are in an “A” configuration, with a middle panel 110 that serves as a back rest angled up from the flat panel 110) as shown in FIG. 5, it can be a chair for meditation or just sitting. In the embodiment of the body wellness board/device 100 that includes the frequency emission device (e.g., blue tooth speaker(s)), the blue-tooth speaker(s) 190 receive(s) signals from the signal generating device 200 such as a mobile device (e.g., iPhone, Android phone, iPad), and emit(s) a frequency or frequency that is transmitted to the board 100 surrounding the routed speaker slot 180. Board area(s)/vibroacoustic housing 230 surrounding the routed speaker slot 180 and the frequency emission device 190 amplifies sound vibrations emitted from the frequency emission device 190 and transmits them to the user's body thereon.

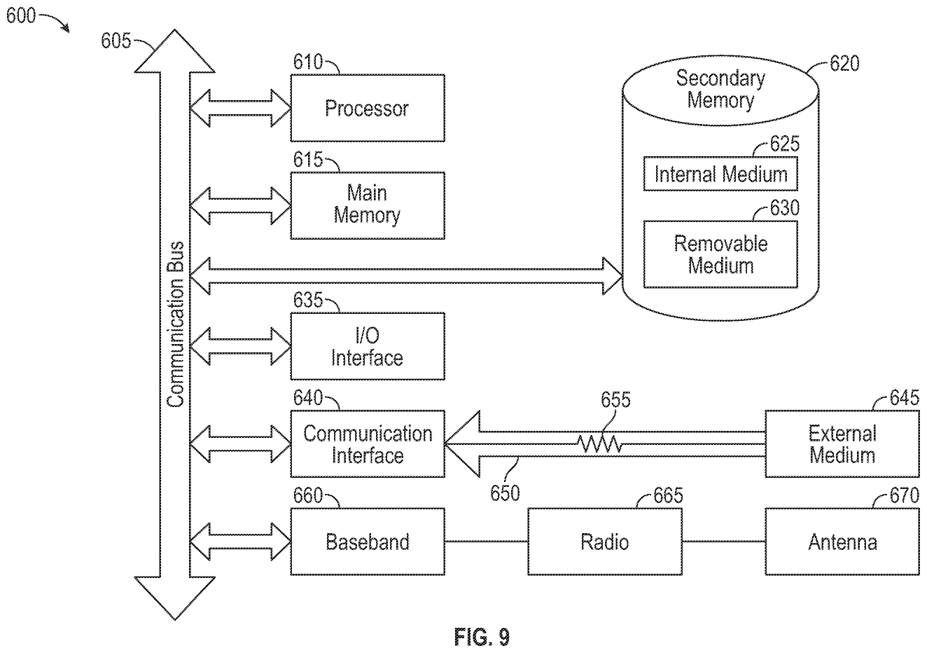

FIG. 9 is a block diagram illustrating an example wired or wireless system 600 that may be used in connection with various embodiments described herein such as, but not limited to, the respective wireless frequency emission device (e.g., blue-tooth speakers) 190 and/or frequency generating device 200 such as a mobile device (e.g., iPhone, Android phone, iPad). For example, system 600 may be used as or in conjunction with one or more of the functions, processes, or methods (e.g., to store and/or execute the application or one or more software modules of the application) described herein. System 600 can be any type of computer or any other processor-enabled device that is capable of wired or wireless data communication. Other computer systems and/or architectures may be also used, as will be clear to those skilled in the art.

System 600 preferably includes one or more processors, such as processor 610. Additional processors may be provided, such as an auxiliary processor to manage input/output, an auxiliary processor to perform floating-point mathematical operations, a special-purpose microprocessor having an architecture suitable for fast execution of signal-processing algorithms (e.g., digital-signal processor), a slave processor subordinate to the main processing system (e.g., back-end processor), an additional microprocessor or controller for dual or multiple processor systems, and/or a coprocessor. Such auxiliary processors may be discrete processors or may be integrated with processor 610. Examples of processors which may be used with system 600 include, without limitation, the Pentium® processor, Core i7® processor, and Xeon® processor, all of which are available from Intel Corporation of Santa Clara, Calif.

Processor 610 is preferably connected to a communication bus 605. Communication bus 605 may include a data channel for facilitating information transfer between storage and other peripheral components of system 600. Furthermore, communication bus 605 may provide a set of signals used for communication with processor 610, including a data bus, address bus, and/or control bus (not shown). Communication bus 605 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, standards promulgated by the Institute of Electrical and Electronics Engineers (IEEE) including IEEE 488 general-purpose interface bus (GPIB), IEEE 696/S-100, and/or the like.

System 600 preferably includes a main memory 615 and may also include a secondary memory 620. Main memory 615 provides storage of instructions and data for programs executing on processor 610, such as one or more of the functions and/or modules discussed herein. It should be understood that programs stored in the memory and executed by processor 610 may be written and/or compiled according to any suitable language, including without limitation C/C++, Java, JavaScript, Perl, Visual Basic, .NET, and the like. Main memory 615 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

Secondary memory 620 may optionally include an internal medium 625 and/or a removable medium 630. Removable medium 630 is read from and/or written to in any well-known manner. Removable storage medium 230 may be, for example, a magnetic tape drive, a compact disc (CD) drive, a digital versatile disc (DVD) drive, other optical drive, a flash memory drive, and/or the like.

Secondary memory 620 is a non-transitory computer-readable medium having computer-executable code (e.g., disclosed software modules) and/or other data stored thereon. The computer software or data stored on secondary memory 620 is read into main memory 615 for execution by processor 610.

In alternative embodiments, secondary memory 620 may include other similar means for allowing computer programs or other data or instructions to be loaded into system 600. Such means may include, for example, a communication interface 640, which allows software and data to be transferred from external storage medium 645 to system 600. Examples of external storage medium 645 may include an external hard disk drive, an external optical drive, an external magneto-optical drive, and/or the like. Other examples of secondary memory 620 may include semiconductor-based memory, such as programmable read-only memory (PROM), erasable programmable read-only memory (EPROM), electrically erasable read-only memory (EEPROM), and flash memory (block-oriented memory similar to EEPROM).

As mentioned above, system 600 may include a communication interface 640. Communication interface 640 allows software and data to be transferred between system 600 and external devices (e.g. printers), networks, or other information sources. For example, computer software or executable code may be transferred to system 600 from a network server via communication interface 640. Examples of communication interface 640 include a built-in network adapter, network interface card (NIC), Personal Computer Memory Card International Association (PCMCIA) network card, card bus network adapter, wireless network adapter, Universal Serial Bus (USB) network adapter, modem, a wireless data card, a communications port, an infrared interface, an IEEE 1394 fire-wire, and any other device capable of interfacing system 600 with a network or another computing device. Communication interface 640 preferably implements industry-promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 640 are generally in the form of electrical communication signals 655. These signals 655 may be provided to communication interface 640 via a communication channel 650. In an embodiment, communication channel 650 may be a wired or wireless network, or any variety of other communication links. Communication channel 650 carries signals 655 and can be implemented using a variety of wired or wireless communication means including wire or cable, fiber optics, conventional phone line, cellular phone link, wireless data communication link, radio frequency (“RF”) link, or infrared link, just to name a few.

Computer-executable code (e.g., computer programs, such as the disclosed application, or software modules) is stored in main memory 615 and/or secondary memory 620. Computer programs can also be received via communication interface 640 and stored in main memory 615 and/or secondary memory 620. Such computer programs, when executed, enable system 600 to perform the various functions of the disclosed embodiments as described elsewhere herein.

In this description, the term “computer-readable medium” is used to refer to any non-transitory computer-readable storage media used to provide computer-executable code and/or other data to or within system 600. Examples of such media include main memory 615, secondary memory 620 (including internal memory 625, removable medium 630, and external storage medium 645), and any peripheral device communicatively coupled with communication interface 640 (including a network information server or other network device). These non-transitory computer-readable media are means for providing executable code, programming instructions, software, and/or other data to system 600.

In an embodiment that is implemented using software, the software may be stored on a computer-readable medium and loaded into system 600 by way of removable medium 630, I/O interface 635, or communication interface 640. In such an embodiment, the software is loaded into system 600 in the form of electrical communication signals 655. The software, when executed by processor 610, preferably causes processor 610 to perform one or more of the processes and functions described elsewhere herein.

In an embodiment, I/O interface 635 provides an interface between one or more components of system 600 and one or more input and/or output devices. Example input devices include, without limitation, sensors, keyboards, touch screens or other touch-sensitive devices, biometric sensing devices, computer mice, trackballs, pen-based pointing devices, and/or the like. Examples of output devices include, without limitation, other processing devices, cathode ray tubes (CRTs), plasma displays, light-emitting diode (LED) displays, liquid crystal displays (LCDs), printers, vacuum fluorescent displays (VFDs), surface-conduction electron-emitter displays (SEDs), field emission displays (FEDs), and/or the like. In some cases, an input and output device may be combined, such as in the case of a touch panel display (e.g., in a smartphone, tablet, or other mobile device).

System 600 may also include one or more optional wireless communication components that facilitate wireless communication over a voice network and/or a data network. The wireless communication components comprise an antenna system 670, a radio system 665, and a baseband system 660. In system 600, radio frequency (RF) signals are transmitted and received over the air by antenna system 670 under the management of radio system 665.

In an embodiment, antenna system 670 may comprise one or more antennae and one or more multiplexors (not shown) that perform a switching function to provide antenna system 670 with transmit and receive signal paths. In the receive path, received RF signals can be coupled from a multiplexor to a low noise amplifier (not shown) that amplifies the received RF signal and sends the amplified signal to radio system 665.

In an alternative embodiment, radio system 665 may comprise one or more radios that are configured to communicate over various frequencies. In an embodiment, radio system 665 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from radio system 665 to baseband system 660.

If the received signal contains audio information, then baseband system 660 decodes the signal and converts it to an analog signal. Then the signal is amplified and sent to a speaker. Baseband system 660 also receives analog audio signals from a microphone. These analog audio signals are converted to digital signals and encoded by baseband system 660. Baseband system 660 also encodes the digital signals for transmission and generates a baseband transmit audio signal that is routed to the modulator portion of radio system 665. The modulator mixes the baseband transmit audio signal with an RF carrier signal, generating an RF transmit signal that is routed to antenna system 670 and may pass through a power amplifier (not shown). The power amplifier amplifies the RF transmit signal and routes it to antenna system 670, where the signal is switched to the antenna port for transmission.

Baseband system 660 is also communicatively coupled with processor 610, which may be a central processing unit (CPU). Processor 610 has access to data storage areas 615 and 620. Processor 610 is preferably configured to execute instructions (i.e., computer programs, such as the disclosed application, or software modules) that can be stored in main memory 615 or secondary memory 620. Computer programs can also be received from baseband processor 660 and stored in main memory 610 or in secondary memory 620, or executed upon receipt. Such computer programs, when executed, enable system 600 to perform the various functions of the disclosed embodiments.

The figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. A body wellness device, comprising:
a plurality of rigid panels;
one or more hinges coupling the rigid panels together whereby the plurality of rigid panels are foldable in an accordion style fashion into at least a flat coplanar configuration, a chair configuration, and carry configuration.

2. The body wellness device of claim 1, wherein the plurality of rigid panels include three tri-fold panels.

3. The body wellness device of claim 1, wherein the plurality of rigid panels are wood panels.

4. The body wellness device of claim 1, wherein the plurality of rigid panels include a rigid surface configured for a user to one or more of practice movements upon, sit upon, and recline upon.

5. The body wellness device of claim 1, wherein the plurality of rigid panels include a mat surface configured for a user to one or more of practice movements upon, sit upon, and recline upon.

6. The body wellness device of claim 1, further including a handle configured to be used to carry the body wellness device.

7. The body wellness device of claim 1, wherein the plurality of rigid panels include a lateral side and a slot whereby when the plurality of rigid panels are in the carry configuration, the plurality of slots overlap each other to form a single handle along the lateral side.

8. The body wellness device of claim 1, wherein the plurality of rigid panels include one or more slots configured to receive respective wireless frequency emission devices.

9. The body wellness device of claim 8, wherein the plurality of rigid panels include one or more wireless frequency emission devices received within the one or more slots, a vibroacoustic housing surrounding the one or more slots, and the one or more wireless frequency emission devices are configured to receive signals from a signal generating device and transmit one or more vibration frequencies to the user via the vibroacoustic housing surrounding the one or more slots.

9. The body wellness device of claim 8, further including one or more rigid inserts received within the one or more slots to cover the one or more slots.

10. The body wellness device of claim 1, wherein the plurality of rigid panels include one or more wireless frequency emission devices, a vibroacoustic housing surrounding the wireless frequency emission devices, and the one or more wireless frequency emission devices are configured to receive signals from a signal generating device and transmit one or more vibration frequencies to the user via the vibroacoustic housing surrounding the one or more wireless frequency emission devices.

11. The body wellness device of claim 9, further including one or more electromagnetic frequency protectors covering the one or more wireless frequency emission devices.

12. A method of using the body wellness device of claim 1, comprising:
deploying the plurality of rigid panels in an accordion style fashion by pivoting the plurality of rigid panels relative to each other into at least the flat coplanar configuration, the chair configuration, and the carry configuration.

13. The method of claim 12, wherein deploying the plurality of rigid panels includes deploying the plurality of rigid panels into the chair configuration where one of the plurality of rigid panels lies flat on a support surface, an adjacent one of the plurality of rigid panels, which forms a back support, is inclined at an angle relative to the flat rigid panel, and a further of the plurality of rigid panels forms an “A” configuration with the adjacent rigid panel with respect to the support surface.

14. The method of claim 12, wherein the plurality of rigid panels include a lateral side and a slot whereby when the plurality of rigid panels are in the carry configuration, the plurality of slots overlap each other to form a single handle along the lateral side, and deploying the plurality of rigid panels includes deploying the plurality of rigid panels into the carry configuration where the plurality of rigid panels stack together so that the plurality of slots overlap each other to form a single handle along the lateral side.

15. The method of claim 12, wherein the plurality of rigid panels include one or more slots and a vibroacoustic housing surrounding the one or more slots, and the method further comprising receiving one or more wireless frequency emission devices within the one or more slots, receiving signals by the one or more wireless frequency emission devices from a signal generating device, and transmitting one or more vibration frequencies to the user via the vibroacoustic housing surrounding the one or more slots.

16. The method of claim 12, wherein the plurality of rigid panels include one or more one or more wireless frequency emission devices and a vibroacoustic housing surrounding the one or more wireless frequency emission devices, and the method further comprising receiving signals by the one or more wireless frequency emission devices from a signal generating device, and transmitting one or more vibration frequencies to the user via the vibroacoustic housing surrounding the one or more slots.
